(19) United States
(12) Patent Application Publication
(10) Pub. No.: US 2006/0102181 A1
(43) Pub. Date: May 18, 2006

(54) Oxygen concentrator with variable temperature and pressure sensing control means

(75) Inventors: Norman R. McCombs, Tonawanda, NY (US); Robert Bosinski, West Seneca, NY (US); Michael R. Valvo, East Aurora, NY (US); Michael A. Chimiak, Williamsville, NY (US)

Correspondence Address: HISCOCK & BARCLAY, LLP, 2000 HSBC PLAZA, ROCHESTER, NY, 14604-2404, US

(73) Assignee: AirSep Corporation

(21) Appl. No.: 11/247,101

(22) Filed: Oct. 11, 2005

Related U.S. Application Data
(60) Provisional application No. 60/617,833, filed on Oct. 12, 2004.
(60) Provisional application No. 60/669,323, filed on Apr. 7, 2005.

(51) Int. Cl. A61M 16/00; A62B 7/04

(52) U.S. Cl. 128/204.26; 128/204.18; 128/204.21

(57) ABSTRACT

An oxygen concentrator for delivering consistent doses of an oxygen concentrated gas to a user by adjusting the delivery time according to the operating pressure and/or temperature of the gas at the time of delivery.

### CROSS-REFERENCE TO RELATED APPLICATIONS

This application claims priority from U.S. Provisional Patent Application Ser. No. 60/617,833, filed Oct. 12, 2004, and U.S. Provisional Patent Application Ser. No. 60/669,323, filed Apr. 7, 2005.

### FIELD OF THE INVENTION

This invention relates to the production of gases and the regulation of their flow and, more specifically, the production of oxygen enriched gases and their delivery in pulse doses.

### BACKGROUND OF THE INVENTION

Gas flow regulators are well known to be used in conjunction with gas supply sources such as high pressure oxygen tanks or other similar oxygen sources to supply oxygen enriched gases, for example, to persons requiring supplemental oxygen. Oxygen control devices have been developed that conserve such an oxygen supply by limiting its release only during useful times such as, for example, during the inhalation period of the person's breathing cycle. In such a device, drops in pressure are caused by inhalation which, in turn, activates the oxygen flow.

It also is known that the only air or oxygen usefully absorbed by the lungs is that oxygen inhaled at the initial or effective stage of inhalation or inspiration. The air or oxygen inhaled in the latter stage of inhalation is usually exhaled before it can be absorbed by the lungs. To take advantage of this phenomenon, a device may conserve oxygen supplies even more by actuating the flow of gas upon initial inhalation but also terminating the flow of oxygen after the effective stage. It is known, with such devices, to control the effective flow rate of the oxygen, according to the user's needs, by increasing or decreasing the activation time during each inhalation cycle.

One such combination pressure regulator and conservation device is disclosed in co-owned U.S. Pat. No. 6,427,690 to McCombs et al, issued Aug. 6, 2002, the entire disclosure of which is incorporated by reference herein, which may conveniently be positioned directly on an oxygen tank (containing oxygen or an oxygen mixture in gas or liquid form), or connected to the wall outlet of a master oxygen system, for connection directly to the tank or outlet. Contained within the device is an oxygen pressure regulator, a power supply or external power supply connection and a control circuit to control the effective dose of oxygen by control of the interval(s) and time(s) of the oxygen flow during every inhalation stage, during selectable, alternate inhalation cycles, or by a continuous supply of oxygen.

The conservation device may contain a first chamber to control the pressure of the supplied oxygen by a regulator spring and piston and may also contain a second or oxygen volume chamber in fluid connection with the first chamber. The second chamber is provided to maintain a predefined volume or “bolus” of oxygen at the pre-set pressure, and from which the oxygen is delivered through a tube to a user upon actuation of a valve operated by a control circuit. To actuate the valve in response to inhalation by the user, as disclosed for example in the foregoing patent, the control circuit includes a pressure sensing transducer that will sense a reduction in pressure caused by the inhalation and thus open the valve for a pre-programmed or otherwise suitable time.

In addition to the conservation device disclosed in U.S. Pat. No. 6,427,690, a portable oxygen concentrator has also been developed which operates on pressure swing adsorption, or PSA, principles and includes an integral oxygen conservation device, as disclosed in co-owned U.S. Pat. No. 6,764,534, McCombs et al, issued Jul. 20, 2004, the entire disclosure of which is incorporated by reference. Furthermore, such an oxygen concentrator described in that patent is able to deliver, at the initial stage of inhalation, a product gas with a high oxygen concentration (e.g., up to about 95% oxygen) produced by the PSA components of the concentrator, equivalent therapeutically to continuous flow rates of at least up to 5 liters per minute (LPM).

The desired mode of operation is determined by positioning a mode control switch to the desired operating mode position. If the conservation device is a separate device, it is attached either to an oxygen tank or the outlet of a PSA apparatus, and the valve on the oxygen supply tank is then opened or the PSA apparatus turned on. In the normal intermittent operating mode, selector switches are used to select one of several operating settings to indicate the equivalent flow rate of the supplied oxygen, e.g., from 1-5 LPM. The oxygen delivery device, such a nose cannula, is then attached by its connecting tube to the outlet on the conservation device

### SUMMARY OF THE INVENTION

The present invention provides an apparatus that is able to produce a product gas having a high concentration of a desired product gas or gases, such as oxygen, with the ability to control more accurately the amount of product gas to a user only on initiation of demand. This invention comprises a compressed product gas (e.g. oxygen) source or other such product gas producing means, such as a pressure swing adsorption (PSA) apparatus or vacuum pressure swing adsorption apparatus (VPSA), and a delivery control assembly to determine the length of time to supply the more accurate amount of product gas to the user by reference to certain operating properties of the apparatus.

As applied to an oxygen producing device, for example, the delivery control assembly serves two primary functions. First, since most oxygen normally inhaled is immediately exhaled and unused, the delivery control assembly provides a pulse dose of oxygen-rich gas only when it will be most efficiently utilized by the person inhaling it, thus minimizing unnecessary waste of the oxygen-rich product gas. This more efficient use of the oxygen supplied is very advantageous in minimizing the capacity requirements of the oxygen source, such as a compressed bottle or PSA apparatus. Reduced capacity requirements may translate to smaller, lighter, quieter and less expensive oxygen-rich gas production devices.

Second, the delivery control assembly, according to this invention, serves to ensure that its owner receives for any given flow setting a substantially constant quantity of oxygen during every inhalation. Because of the Ideal Gas Law, PV=nRT, it cannot be assumed that this amount will always be constant because the number of oxygen molecules in each dose will depend on the partial pressure of the oxygen enriched gas in the apparatus which, in turn, will depend upon a number of factors, but primarily the pressure and temperature of the gas within the apparatus at the time of inhalation.

This invention uses sensors that read, for example, real-time operating pressures and/or temperatures, and converts the analog outputs of the sensors to digital signals to control the pulse dose through the use of a microprocessor in a micro-electronic control circuit. The control circuit also has means to respond to the initiation of inhalation by the user and produce a digital signal to the microprocessor, which in turn calculates the proper pulse dose duration based on the signal inputs, for example, by the microprocessor accessing pre-programmed data tables. The invention will also be able to correct for temperature and/or pressure variations within the apparatus resulting from a PSA or VPSA operating cycle and administer oxygen gas consistently to a user regardless of when in the operating cycle the inhalation is detected.

### BRIEF DESCRIPTION OF THE DRAWINGS

The above-mentioned and other features and advantages of this invention, and the manner of attaining them, will become apparent and be better understood by reference to the following description of several embodiments of the invention in conjunction with the accompanying drawings, wherein:

FIG. 1 is a schematic view of a Pressure Swing Adsorption (PSA) apparatus in which the invention may be incorporated;

FIG. 2 is a schematic view of a Vacuum Pressure Swing Adsorption (VPSA) apparatus that also may incorporate the invention;

FIG. 3 is a partial schematic view illustrating the control assembly for the first embodiment of the invention;

FIG. 4 is a block diagram of the control circuit for determining the length of the pulse dose based on the point in the operating cycle that inhalation is sensed;

FIG. 5 is a block diagram of the control circuit for a second embodiment of the invention, by which the pulse dose volume may be controlled for variations in temperature and/or pressure;

FIG. 6 is a partial schematic view illustrating the control assembly for the second embodiment of the invention; and

FIGS. 7a-d together form the schematic of a control circuit used for the invention.

Corresponding reference characters indicate corresponding parts throughout the several views. The examples set out herein illustrate certain embodiments of the invention but should not be construed as limiting the scope of the invention in any manner.

### DETAILED DESCRIPTION

The invention described in this application may be used in either a PSA or VPSA apparatus, both of which are well known and described, for example, in U.S. Pat. Nos. 3,564,816; 3,636,679; 3,717,974; 4,802,899; 5,531,807; 5,755,856; 5,871,564; 6,524,370; and 6,764,534, among others. Both a PSA and a VPSA apparatus may include one or more adsorbers, each having a fixed sieve bed of adsorbent material to fractionate at least one constituent gas from a gaseous mixture by adsorption into the bed, when the gaseous mixture from a feed stream is sequentially directed through the adsorbers in a co-current direction. While one adsorber performs adsorption, another adsorber is purged of its adsorbed constituent gas. In a PSA apparatus, the purging is performed by part of the product gas being withdrawn from the first or producing adsorber and directed through the other adsorber in a counter-current direction. In a VPSA apparatus, the purging primarily is performed by a vacuum produced at the adsorber inlet to draw the purged gas from the adsorber Once the other adsorber is purged, the feed stream at a preset time is then directed to the other adsorber in the co-current direction, so that the other adsorber performs adsorption. The first adsorber is then purged either simultaneously, or in another timed sequence if there are more than two adsorbers, all of which will be understood from a reading of the above described patents.

When, for example, such an apparatus is used to produce a high concentration of oxygen from ambient air for use in various applications, whether medical, industrial or commercial, air enters the apparatus typically containing about 78% nitrogen, 21% oxygen, 0.9% argon and a variable amount of water vapor. Principally, most of the nitrogen is removed by the apparatus to produce the product gas which, for medical purposes, for example, typically may contain at least about 80% and up to about 95% oxygen.

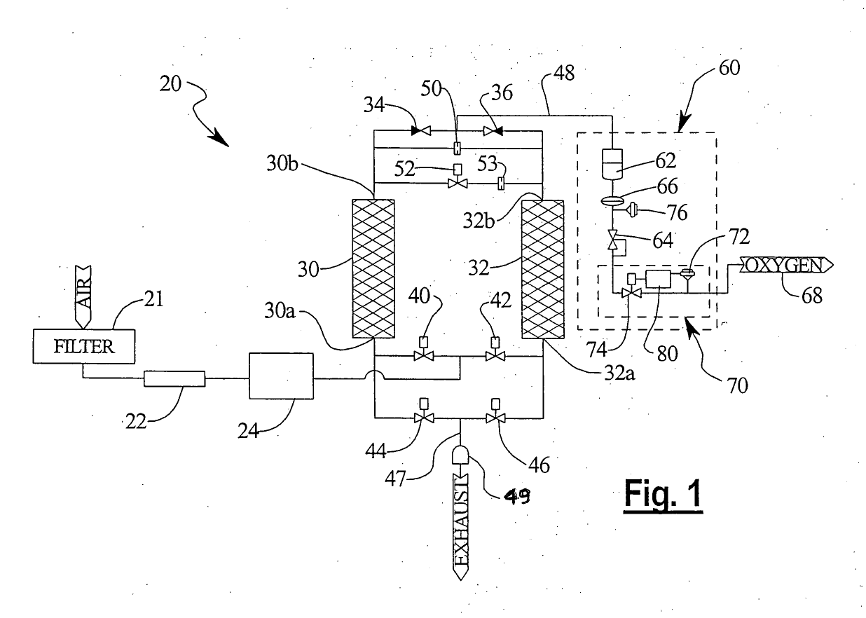

Referring to FIG. 1, ambient air is supplied to a PSA apparatus 20 through a filtered intake 21 and an intake resonator 22 to decrease the noise from the intake of the ambient air feed stream. The feed stream continues from the resonator 22 and is moved by a feed air compressor/heat exchanger assembly 24 alternatively to the first and second adsorbers 30, 32 through feed valves 40 and 42, respectively.

When the feed stream alternatively enters inlets 30a, 32a of adsorbers 30, 32 in a co-current direction, the respective adsorber fractionates the feed stream into the desired concentration of product gas. The adsorbent material used for the beds to separate nitrogen from the ambient air may be a synthetic zeolite or other known adsorber material having equivalent properties.

The substantial or usable portion of the oxygen enriched product gas generated from the ambient air flowing in the co-current direction sequentially in each one of the adsorbers 30, 32 is directed through the outlet 30b, 32b and check valve 34, 36 of the corresponding adsorber to a product manifold 48 and then to a delivery control assembly 60, as will be described. The balance of the product gas generated by each adsorber is timed to be diverted through a purge orifice 50, a properly timed equalization valve 52 and an optional flow restrictor 53 to flow through the other adsorber 30 or 32 in the counter-current direction from the respective outlet 30b, 32b and to the respective inlet 30a, 32a of the other adsorber to purge the adsorbed, primarily nitrogen, gases. The counter-current product gas and purged gases then are discharged to the atmosphere from the adsorbers through properly timed waste valves 44, 46, common waste line 47 and a sound absorbing muffler 49.

The control assembly 60, to which the usable portion of the produced gas is directed, typically includes a mixing tank 62 which also may be filled with synthetic zeolite and serves as a reservoir to store product oxygen before delivery to the user through an apparatus outlet 68 in the pulse dose mode, a pressure sensor 76 to monitor the pressure of the product gas at the mixing tank 62 (normally, for example, to monitor for extreme pressure levels and activate a warning signal), a piston-type pressure control regulator 64 to regulate the product gas pressure to be delivered to the user, an optional bacteria filter 66, and an oxygen delivery system 70 including a pulse dose transducer 72, the conservation unit 80 to be described, and a flow control valve 74. Delivery of the PSA generated oxygen concentrated gas from the mixing tank 62 to the user is controlled by the delivery system 70 as will be described.

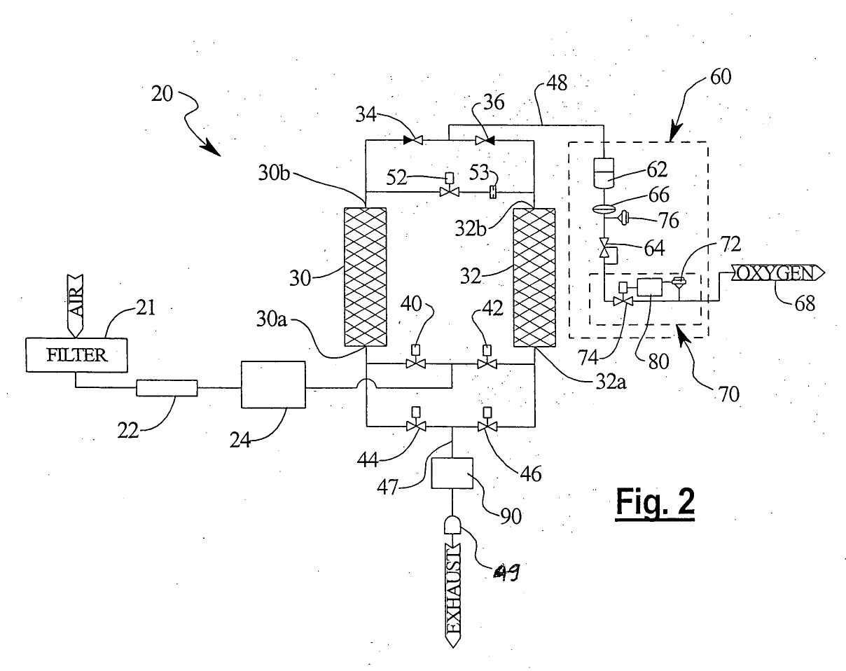

A VPSA apparatus as schematically shown in FIG. 2 operates in similar fashion as the PSA apparatus of FIG. 1, except that the purge orifice 50 may be eliminated. In its stead, a vacuum pump 90 is provided in the common waste line 47 to draw the waste nitrogen alternately from each of adsorber beds 30, 32 upon the timed opening of the respective waste valve 44, 46. The cycling of ambient air and operation of the feed and waste valves to produce the oxygen enriched product gas, as well as of supply of product gas to mixing tank 62 and the delivery of the product gas by conservation unit 80, otherwise are as described with respect to FIG. 1.

As described earlier, a conservation device delivers, when the patient inhales, a consistent and specific pulse dose of oxygen to the patient at preset times depending on the selected flow setting of the device and equivalent to a continuous flow rate. The product gas delivery pressure, as set by a pressure regulator, e.g., 64, together with the preset open time for an oxygen delivery demand valve, which may be a solenoid actuated flow control valve 74 as earlier described, generally determines the volume of the product gas delivered to the user. This technique, to open upon inhalation the demand valve for a certain amount of time to deliver the desired dose, may be used with cylinders of oxygen and in PSA or VPSA oxygen concentrators.

A pressure regulator is known in the prior art to be necessary when a conservation device is used with oxygen cylinders and with oxygen concentrators. Whatever the pressure in an oxygen tank, the regulator regulates the pressure down to approximately 20 psig to obtain a consistent pulse dose as the cylinder depressurizes over time. In a PSA apparatus, the cycle pressure can vary, e.g., from about 15 to about 26 psig, and the regulator regulates the pressure at the demand valve, e.g., to approximately 10 psig. Similarly, the cycle pressure for a VPSA may vary e.g., from about −25 to about 10 psig and regulated at the demand valve to about 3 psig.

Additionally, the actual amount of oxygen to be delivered to a user of the apparatus will be a function of other factors, including the length of time that a valve is open, the operational temperature of the gas at the time it is being supplied and the breathing rate of the user. For example, at a higher temperature, less oxygen will be delivered to a user for any given period of time. Similarly, less oxygen will be delivered to the user at lower pressures caused by, among other things, more rapid breathing rates that will affect the product gas pressure. Unlike the known prior art, the invention described here comprises an oxygen concentrator 20 that is able to control the pulse dose time in order to deliver a substantially consistent and predetermined quantity of oxygen based operating pressures and/or temperatures, as opposed to fixed, predetermined delivery times in which the actual quantity of oxygen will vary based on the Ideal Gas Law.

According to the invention, the pulse dose may be controlled based on the monitoring of a specific system property or a combination of system properties and by these means eliminate the necessity of a pressure regulator that otherwise adds weight to the apparatus. In one embodiment of this invention, the length of the pulse dose to deliver the desired quantity of oxygen is dependent on the pre-calculated and predictable system pressure at the time inhalation starts. In a second embodiment, the length of the pulse dose is determined from the measurement at inhalation of the actual temperature and/or actual pressure of the product gas preferably but not necessarily at or near the mixing tank 62.

The first embodiment of this invention takes advantage of that fact that the amount of oxygen that is delivered by the invention is a function volume pressure at the mixing tank 62 which can be pre-measured during manufacture of the apparatus and then “predicted” during use in the various stages of the operating cycle of the PSA or VPSA, thereby eliminating the need for a pressure regulator. An apparatus that does not need a pressure regulator is highly useful in the effort to make the apparatus as small and light as possible. In this embodiment, a prescribed and consistent dose of oxygen can be delivered by controlling the length of time the demand valve is open at the exact point in the operating cycle when inhalation is sensed. According to this embodiment, pressure sensor transducer 84 may be used to activate a warning signal if the apparatus is not functioning normally, but need not be used to determine the length of the pulse dose.

For example, a PSA with two adsorber beds may have a pressure swing adsorption cycle with an overall time lapse of 17 seconds, or a sub-cycle of about 8.5 seconds for each bed during that bed's oxygen producing phase. By selecting a time interval of 0.85 seconds, the oxygen producing sub-cycle for each operating bed may be divided into 10 different cycle points. The following chart, TABLE 1, shows the variation of the system pressure in the two-bed PSA apparatus over the 17 seconds required to cycle both beds through their oxygen producing sub-cycles. The system pressures versus time as illustrated below are consistently and repeatedly reproduced throughout the cyclical operation of the PSA apparatus.

[image]

To determine the actuating time of the demand valve 74, the pressure at the mixing tank 62, from which the pulse dose volume of oxygen concentrated product gas is delivered to the user, may be divided, for example, into 5 ranges that encompass the measured volume pressure variation range: <21 psig, 21-23.9 psig, 24-26.9 psig, 27-29.9 psig, and >30 psig. Using these time and pressure ranges, a data table is generated for each of the ten selected points in the operating cycle based on an initial nominal time of 200 milliseconds for each point. Therefore, when the demand valve is opened for the 200 ms nominal time to deliver a pulse dose, the actual pulse dose volume, based on the cycle point at which the valve was opened and pulse dose volume pressure, are all measured. If, for example, the device were set to produce a desired pulse dose volume of 26.25 ml and the measured pulse dose volume for that cycle point were to be 24 ml (9% less than the desired 26.25 ml), the time for that cycle point would be changed from 200 ms to 218 ms and the valve open time would be adjusted accordingly. To complete the data table for each desired setting, this process is continued until the correct time for each of the ten cycle points is determined. As seen in TABLE 2, where the flow setting is equivalent to 3 LPM of continuous oxygen supply, a final data table listing the calculated demand valve open times for each of the ten cycle points would read as follows.

TABLE 2

| | Actual Volume Pressure (psig) | | | | |
|---|---|---|---|---|---|
| Points in Cycle Time | <21 | 21-23.9 | 24-26.9 | 27-29.9 | >30 |
| 0-0.85 or 8.5-9.35 | 263 ms | 215 ms | 198 ms | 198 ms | 173 ms |
| 0.85-1.7 or 9.35-10.2 | 228 ms | 218 ms | 207 ms | 200 ms | 186 ms |
| 1.7-2.55 or 10.2-11.05 | 219 ms | 217 ms | 216 ms | 185 ms | 176 ms |
| 2.55-3.4 or 11.05-11.9 | 215 ms | 213 ms | 190 ms | 185 ms | 175 ms |
| 3.4-4.25 or 11.9-12.75 | 208 ms | 205 ms | 203 ms | 180 ms | 173 ms |
| 4.25-5.1 or 12.75-13.6 | 217 ms | 196 ms | 198 ms | 186 ms | 180 ms |
| 5.1-5.95 or 13.6-14.45 | 220 ms | 210 ms | 192 ms | 187 ms | 198 ms |
| 5.95-6.8 or 14.45-15.3 | 225 ms | 204 ms | 189 ms | 186 ms | 170 ms |
| 6.8-7.65 or 15.3-16.15 | 228 ms | 221 ms | 198 ms | 180 ms | 170 ms |
| 7.65-8.5 or 16.15-17.0 | 230 ms | 219 ms | 207 ms | 188 ms | 190 ms |

In the same manner as the pulse dose times are determined in TABLE 2, the technique is used for creating other data tables of calculated pulse dose times for other selectable flow settings used in the apparatus.

An alternate apparatus having a cycle time of about 12 seconds and three flow rates as illustrated in TABLE 3, made according to the invention described in co-pending provisional application by McCombs et al., Mini-Portable Oxygen Concentrator, Ser. No. 60/617,834, filed Oct. 12, 2004, the entire disclosure of which is incorporated by reference, may have look-up tables for valve open times in milliseconds, as shown in TABLE 4.

[image]

TABLE 4
Valve Open Times (Milliseconds)

| | Pressure (psig) | | | | | | | | | |
|---|---|---|---|---|---|---|---|---|---|---|
| Cycle Time (sec) | <16 | 16-16.9 | 17-17.9 | 18-18.9 | 19-20.9 | 21-22.9 | 23-24.9 | 25-26.9 | 27-29 | >29 |

1 LPM

| | | | | | | | | | | |
|---|---|---|---|---|---|---|---|---|---|---|
| 0-2.09 | 85 | 85 | 85 | 85 | 85 | 85 | 85 | 85 | 85 | 85 |
| 2.1-3.09 | 90 | 90 | 90 | 90 | 90 | 90 | 90 | 90 | 90 | 90 |
| 3.1-4.09 | 90 | 90 | 90 | 90 | 90 | 90 | 90 | 90 | 90 | 90 |
| 4.1-5.09 | 85 | 85 | 85 | 85 | 85 | 85 | 85 | 85 | 85 | 85 |
| 5.1-6.09 | 80 | 80 | 80 | 80 | 80 | 80 | 80 | 80 | 80 | 80 |
| 6.1-8.19 | 85 | 85 | 85 | 85 | 85 | 85 | 85 | 85 | 85 | 85 |
| 8.2-9.19 | 90 | 90 | 90 | 90 | 90 | 90 | 90 | 90 | 90 | 90 |
| 9.2-10.19 | 90 | 90 | 90 | 90 | 90 | 90 | 90 | 90 | 90 | 90 |
| 10.2-11.19 | 85 | 85 | 85 | 85 | 85 | 85 | 85 | 85 | 85 | 85 |
| 11.2-12.2 | 80 | 80 | 80 | 80 | 80 | 80 | 80 | 80 | 80 | 80 |

2 LPM

| | | | | | | | | | | |
|---|---|---|---|---|---|---|---|---|---|---|
| 0-2.09 | 155 | 155 | 150 | 150 | 140 | 130 | 120 | 120 | 120 | 120 |
| 2.1-3.09 | 150 | 150 | 145 | 140 | 140 | 130 | 120 | 120 | 120 | 120 |
| 3.1-4.09 | 155 | 155 | 140 | 140 | 135 | 125 | 120 | 120 | 120 | 120 |
| 4.1-5.09 | 155 | 155 | 150 | 135 | 130 | 120 | 115 | 115 | 115 | 115 |
| 5.1-6.09 | 155 | 155 | 155 | 140 | 130 | 125 | 115 | 115 | 115 | 115 |
| 6.1-8.19 | 155 | 155 | 150 | 150 | 140 | 130 | 120 | 120 | 120 | 120 |
| 8.2-9.19 | 150 | 150 | 145 | 140 | 140 | 130 | 120 | 120 | 120 | 120 |
| 9.2-10.19 | 155 | 155 | 140 | 140 | 135 | 125 | 120 | 120 | 120 | 120 |
| 10.2-11.19 | 155 | 155 | 150 | 135 | 130 | 120 | 115 | 115 | 115 | 115 |
| 11.2-12.2 | 155 | 155 | 155 | 140 | 130 | 125 | 115 | 115 | 115 | 115 |

3 LPM

| | | | | | | | | | | |
|---|---|---|---|---|---|---|---|---|---|---|
| 0-2.09 | 220 | 220 | 220 | 220 | 215 | 210 | 200 | 190 | 190 | 190 |
| 2.1-3.09 | 220 | 220 | 220 | 215 | 210 | 205 | 195 | 185 | 190 | 190 |
| 3.1-4.09 | 220 | 220 | 220 | 220 | 200 | 195 | 190 | 175 | 180 | 180 |
| 4.1-5.09 | 220 | 220 | 220 | 220 | 210 | 190 | 185 | 170 | 175 | 175 |
| 5.1-6.09 | 220 | 220 | 220 | 220 | 210 | 200 | 180 | 180 | 180 | 180 |
| 6.1-8.19 | 220 | 220 | 220 | 220 | 215 | 210 | 200 | 190 | 190 | 190 |
| 8.2-9.19 | 220 | 220 | 220 | 215 | 210 | 205 | 195 | 185 | 190 | 190 |
| 9.2-10.19 | 220 | 220 | 220 | 220 | 200 | 195 | 190 | 175 | 180 | 180 |
| 10.2-11.19 | 220 | 220 | 220 | 220 | 210 | 190 | 185 | 170 | 175 | 175 |
| 11.2-12.2 | 220 | 220 | 220 | 220 | 210 | 200 | 180 | 180 | 180 | 180 |

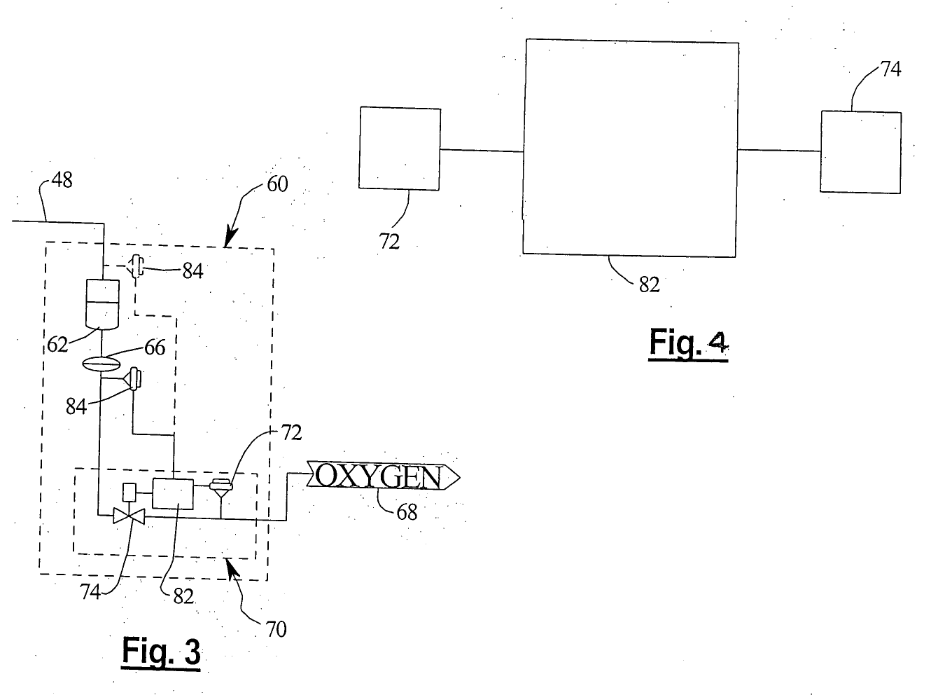

Preferably, and to improve significantly the power efficiency of the apparatus, the compressor/heat exchanger assembly 24 is programmed to operate at a different speed for each flow setting, as for example according to the apparatus disclosed in co-pending provisional application Ser. No. 60/617,834, at speeds of about 1750 rpm for the equivalent continuous flow rate of 1 LPM, about 2500 rpm for the equivalent continuous flow rate of 2 LPM, and about 3200 rpm for the equivalent continuous flow rate of 3 LPM. All of the tables then are stored in the oxygen concentrator's microprocessor 82 to be accessed during use. As microprocessor 82 primarily controls the sequence of operation of all operating components of the apparatus, it inherently contains the information as to the PSA cycle. Because the microprocessor continues to monitor the operating cycle of the PSA, it will integrate the selected flow setting with the predetermined volume pressure at the mixing tank for the cycle point, when inhalation is sensed by the pressure transducer 72, at which point in time, the microprocessor logic consults the data table for the corresponding setting and opens the demand valve 74 for the corresponding length of time listed in the table. A schematic of a control assembly 60 without a pressure regulator according to this embodiment of the present invention is shown in FIG. 3.

The microprocessor 82 is further pre-programmed to include the data of all of the data tables which define the length of time that the demand valve 74 is to remain open, as described above, and as determined by cycle time. Depending on the setting of the apparatus and on receipt of the analog signal from the pulse dose transducer 72, the microprocessor 82 refers to the appropriate data table and then serves to actuate the demand valve 74 according to the factors defined in the data table.

FIG. 4 is a block diagram representing the control circuit according to this embodiment. For illustrative purposes, the figure includes only the pressure transducer 72 to sense inhalation, the microprocessor 82 containing the information for both the point in time of the operating cycle and the look-up table, and the demand valve 74. Generally, the inhalation pressure transducer 72 serves to detect a change in pressure which would indicate the start of the inhalation cycle. Upon sensing inhalation, the inhalation pressure transducer 72 transmits a signal suitable for processing by the microprocessor 82, which in turn accesses its look-up table and signals the demand valve 74 to be actuated for the appropriate length of time. While FIG. 4 provides a block diagram representative of the control circuit according to the present invention, details of the specific circuit elements and microprocessor logic can be determined by those skilled in the art and by reference, for example, to the circuit described in U.S. Pat. No. 6,764,534.

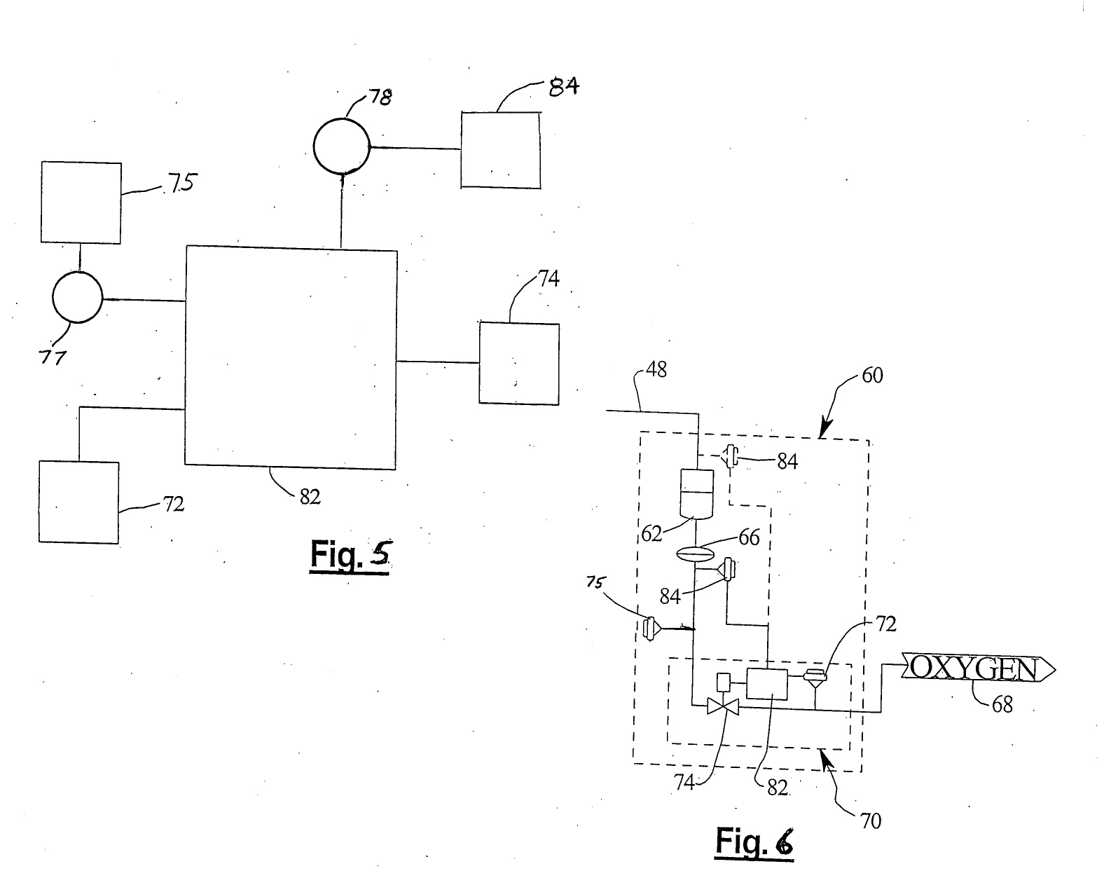

FIG. 5 and FIG. 6 are representative of the control circuit 60 according to a second embodiment of the present invention, by which the actual operating pressures and/or actual operating temperatures are used to determine the dose of oxygen enriched product gas to be delivered to the user. For illustrative purposes, FIG. 5 is a block diagram that includes only the pressure transducer 72 to sense inhalation, a temperature sensing circuit 77 for reading an analog signal of the temperature sensed by a temperature sensor or thermistor 75 at the point of inhalation and converting it to a digital signal, a pressure sensor 84 at the mixing tank the output of pressure sensor 84 if not a digital signal is converted to a digital signal by a pressure sensing circuit 78, microprocessor 82 to read the two digital signals, and control demand valve 74 actuated by the microprocessor 82 in response to pressure transducer 72. Upon detection of inhalation by the pressure transducer 72, the microprocessor 82 reads the digital signals derived from the temperature sensor 75 and pressure sensor 84 respectively. As noted before, these sensors may take their measurements at one of several locations within the apparatus such as, for example, at the outlet or inlet of mixing tank 62, as shown in FIG. 6. It is also possible that the pressure sensor 84 and the temperature sensor 75, although depicted as separate instruments, may be a single monitoring device capable of reading both parameters. Moreover, while FIG. 5 illustrates the possibility that both actual temperature and actual pressure may be used to control the length of the pulse dose, it also is possible according to the invention to control the pulse dose using only one of those parameters, or to use the temperature input in combination with the pre-calculated system pressure inputs as described in the first embodiment.

If, for example, it is desired to use only variations in temperature to control the length of the pulse dose, and as will be described in greater detail below, the microprocessor 82 receives the signal produced by the temperature sensing circuit 77, determines which of temperature range within which the gas is at the time of inhalation, and from the appropriate data table, determines the time that the demand valve needs to remain open to deliver a prescribed amount of oxygen for the selected equivalent flow rate. For this particular embodiment of the present invention, the temperature ranges, three for example, are defined for each of the predetermined number of pressure ranges, as described in the first embodiment, and for each of the selectable settings for the concentrator. Each of the three temperature ranges has a designated time for the demand valve to remain open and otherwise corresponding to the preset valve open times according to the selected flow rate for the apparatus. Additionally, because the device may have, e.g., three selectable flow settings, look-up tables for the times for the demand valve to remain open are also defined specifically for each flow setting.

As can be seen in TABLE 5, which illustrates a concentrator having five flow selector settings, when the temperature sensing circuit reads a temperature less than or equal to about 15° C. for a particular flow setting, the demand valve 74 remains open for a time period as defined for that particular temperature range. This time period is received by the microprocessor from the appropriate data table. If the temperature is greater than about 15° C. and less than about 30° C., the demand valve 74 parameters in the look-up table for that particular temperature range are accessed, and if the temperature is greater than or equal to about 30° C., the demand valve 74 parameters for that particular temperature range are accessed, and so forth. Generally, a temperature increase results in an increase in time the demand valve 74 remains open, or the Pulse Dose Time.

TABLE 5

| Flow Selector | Pulse Dose Time | | |
|---|---|---|---|
| Setting (LPM) | 5-15° C. | 15-30° C. | 30-40° C. |
| 1 | 50 ms | 55 ms | 60 ms |
| 2 | 110 ms | 115 ms | 125 ms |
| 3 | 175 ms | 180 ms | 190 ms |
| 4 | 235 ms | 245 ms | 260 ms |
| 5 | 295 ms | 310 ms | 325 ms |

Of course, TABLE 5 is only functional for a specific pressure range, for example, if the system pressure in the concentrator also is used to regulate flow. Thus, if according to the first embodiment or the second embodiment in which the pressure regulator is eliminated, then it also is useful to create a look-up table for each one of the predetermined number of temperature ranges and each one of the predetermined pressure ranges. This is the case whether the pressures for the pressure ranges are the pre-calculated pressures as in the first embodiment or are actual pressures as measured by the pressure sensor 76. Therefore, additional temperature tables are provided for the other predetermined pressure ranges. The result is a three dimensional matrix of look-up tables for each temperature range, pressure range and flow setting.

All of the information from the tables is stored in the microprocessor to be accessed during use of the apparatus. The microprocessor monitors the selected flow setting and determines the appropriate temperature range of the gas based on the analog signal received by the temperature sensing circuit 77. At the time an inhalation is sensed, the microprocessor logic consults the data table for the corresponding setting and opens the demand valve for the corresponding length of time listed in the table for the appropriate pressure range as detected by the pressure sensor 76.

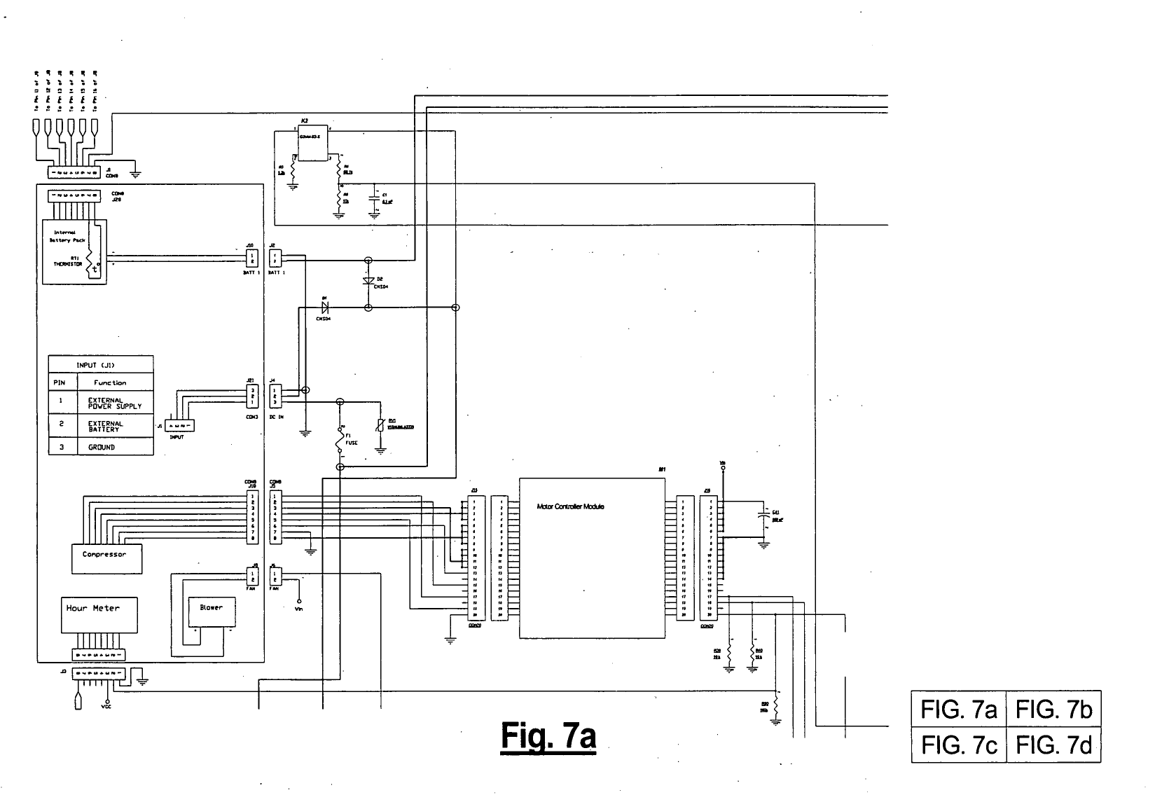

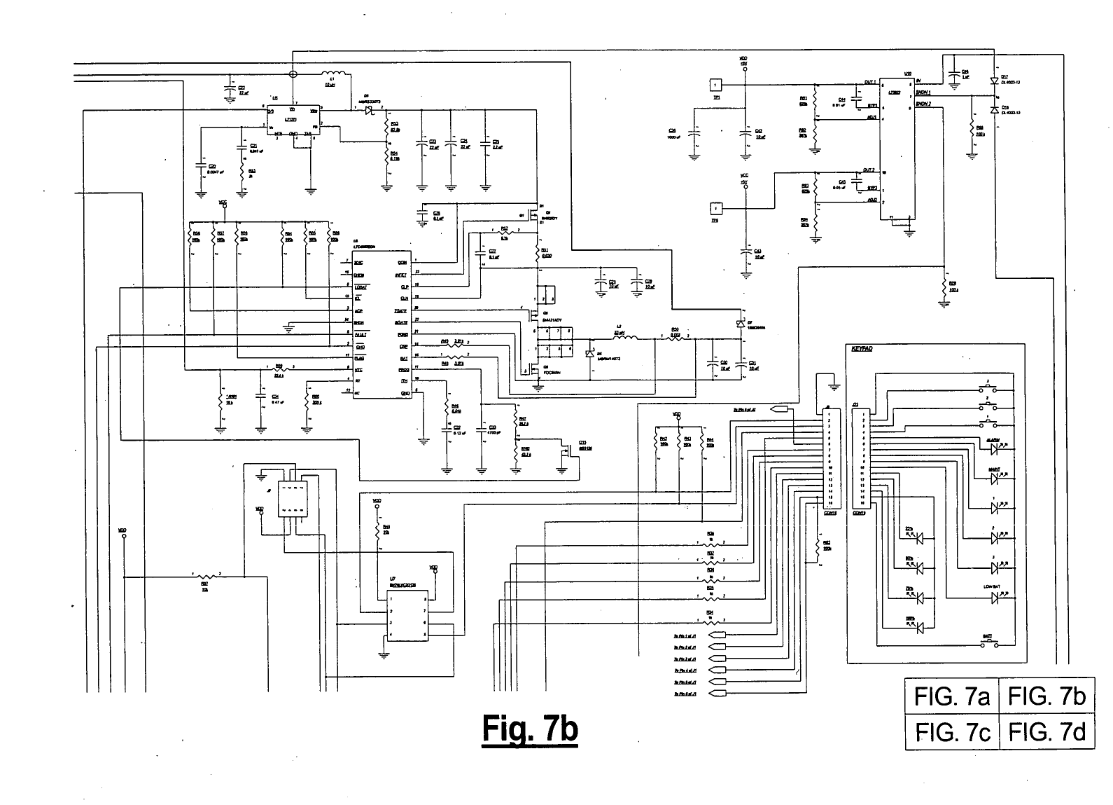

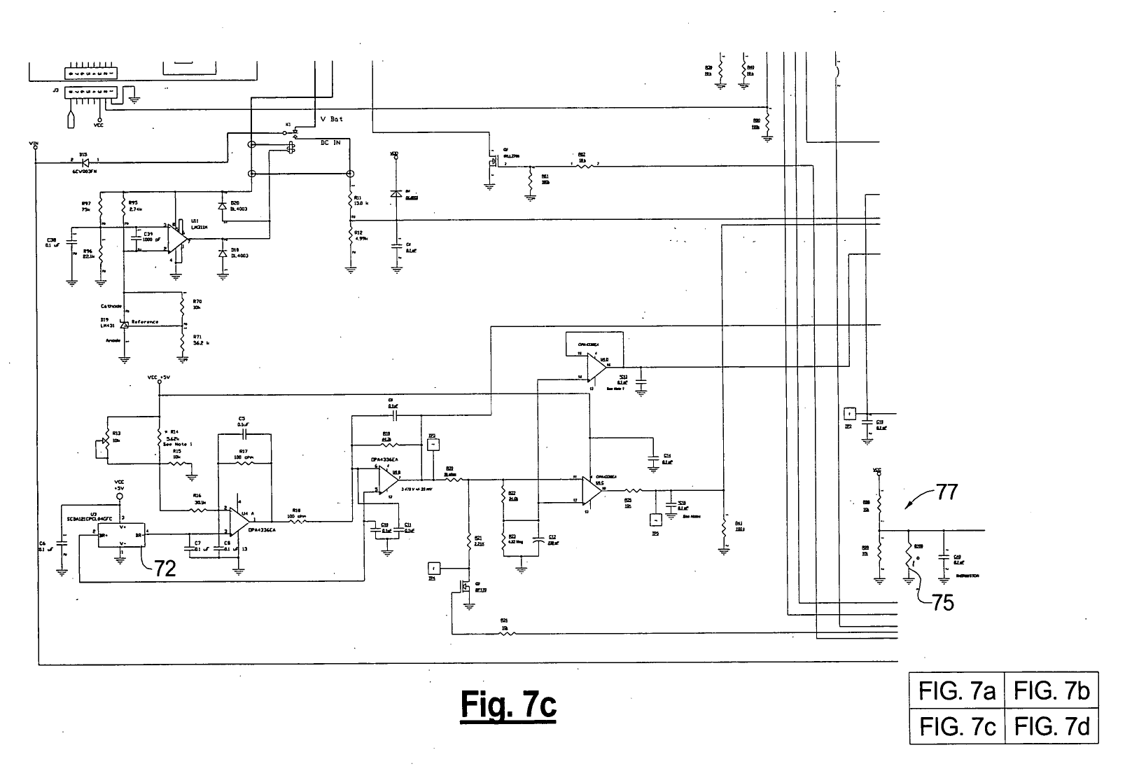

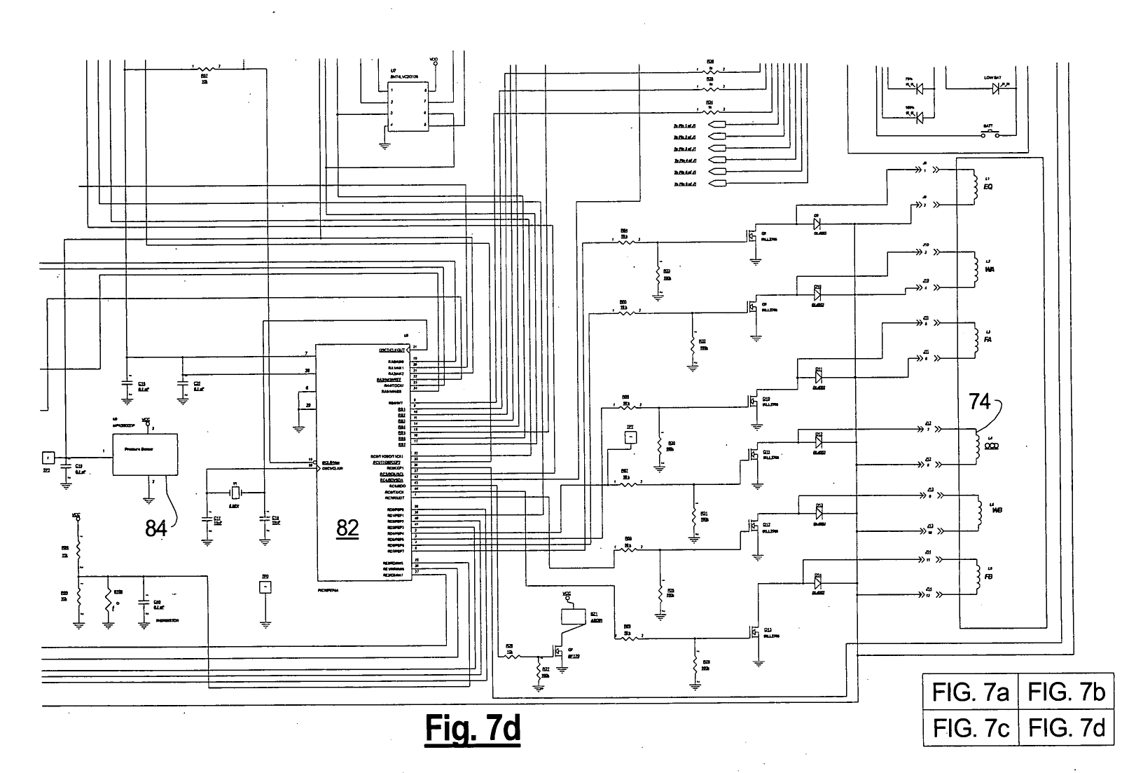

The microprocessor 82 is pre-programmed to contain all of the data tables which define the length of time that the demand valve 74 is to remain open, as described above, for each of the temperature ranges within a given pressure range, or vice versa. Based on the flow selector setting and on receipt of the digital signals derived from the temperature sensor 75 and pressure sensor 84, the microprocessor 82 refers to the appropriate data table which then actuates the demand valve 74 according to the time value listed in the data table. While FIG. 5 provides a block diagram representative of the control circuit according to the second embodiment of the present invention, details of the specific circuit elements and microprocessor logic can be determined by those skilled in the art and by reference, for example, to the circuit described in U.S. Pat. No. 6,764,534, or by reference to FIGS. 7a-d. FIGS. 7a-d, are the four quadrants of the circuit that can be joined by reference to the common elements in the respective quadrants.

One version of the temperature sensing circuit 77, as seen in the schematic in FIGS. 7a-d, is comprised of a resistor divider network that is added to a circuit board (not shown). More particularly, the temperature sensing circuit 77 includes resistor divider network R86, R87, and a 10K negative temperature coefficient thermistor. In operation, the microprocessor 82 reads the voltage that is developed by the temperature sensing circuit 77 every time an inhalation is detected. For example, given a constant electrical current, as unit temperature rises, thermistor resistance also rises, resulting in a higher output voltage, which is read by the microprocessor in the appropriate table to produce the desired dose time.

One version of the pressure sensor 84, not requiring a separate pressure sensing circuit 78 to convert to a digital signal, is presented in the schematic depicted in FIGS. 7a-d and serves to measure gas pressure at the output of the mixing tank and produce a signal which is read by the microprocessor 82 via an analog to digital converter port. The microprocessor 82 is programmed to determine the appropriate pressure range based upon the analog output of the pressure sensor 84 and the tables provided. Additionally, the microprocessor 82 can be used to identify unusually low and high pressure levels at the mixing tank and serve to identify system failures. For example, when the pressure sensor 84 reads a pressure of about 2 psi or lower, the microprocessor 82 signals a system failure. A similar failure is signaled if the system pressure is about 36 psi or higher.

As it has been described that the amount of oxygen administered is a function of pulse dose time as related to the pressure of the oxygen in the system, it should be reasonably clear that oxygen pressure, which would thereby determine pulse dose time, is dependent upon both the ambient conditions, such as pressure and temperature as well as the fluctuations in pressure inherent to a PSA or VPSA apparatus. Therefore, in the more preferred embodiment, the control assembly operation is determined by a cross-referencing of temperature/pressure input with volume pressure.

Because the flow selector settings (in LPM) in principle are common in the previous embodiments, there still remains a human element in deciding the specified pulse dose. In the first embodiment, the data essentially used to calculate pulse dose values are in terms of a correction factor to a nominal pulse dose of 200 ms. In the second embodiment, however, a nominal pulse dose is no longer used as a base point, but instead the dose is determined by the microprocessor calculating the actual pulse dose times based on actual pressure and temperature inputs. Thus, in the second embodiment, the microprocessor 82, continuously receives baseline temperature and pressure information derived from the temperature sensor 75 and pressure sensor 84. It is preferable that these values be constantly be measured as the microprocessor 82 may need to average a relatively short time history of those values to adjust baseline pressures and temperatures over time during use of the apparatus. From this baseline set of values, the microprocessor may know the proper baseline pulse dose from a designated table stored in the microprocessor memory. When the inhalation pressure transducer 72 senses a pressure drop due to inhalation, the microprocessor 82 senses this and reads the volume pressure at that moment in time via the pulse dose transducer 84. This value will allow the microprocessor locate a correction factor from a independent set of tables which are based on the continuously changing volume pressure in a PSA or VPSA cycle, and apply that correction to produce the final required pulse dose.

For example, TABLE 2 was expressed as a series of pulse dose times based on a nominal value of 200 ms. However, these values could simply be expressed as a multiplication factor and theoretically applied to any nominal value as shown in TABLE 6 below:

TABLE 6

| | Actual Volume Pressure (psig) | | | | |
|---|---|---|---|---|---|
| Points in Cycle Time | <21 | 21-23.9 | 24-26.9 | 27-29.9 | >30 |
| 0-0.85 or 8.5-9.35 | 1.315 | 1.075 | .99 | .99 | .865 |
| 0.85-1.7 or 9.35-10.2 | 1.14 | 1.09 | 1.035 | 1 | .93 |
| 1.7-2.55 or 10.2-11.05 | 1.095 | 1.085 | 1.08 | .925 | .88 |
| 2.55-3.4 or 11.05-11.9 | 1.075 | 1.065 | .95 | .925 | .875 |
| 3.4-4.25 or 11.9-12.75 | 1.04 | 1.025 | 1.015 | .9 | .865 |
| 4.25-5.1 or 12.75-13.6 | 1.085 | .98 | .99 | .93 | .9 |
| 5.1-5.95 or 13.6-14.45 | 1.1 | 1.05 | .96 | .935 | .99 |
| 5.95-6.8 or 14.45-15.3 | 1.125 | 1.02 | .945 | .93 | .85 |
| 6.8-7.65 or 15.3-16.15 | 1.14 | 1.105 | .99 | .9 | .85 |
| 7.65-8.5 or 16.15-17.0 | 1.15 | 1.095 | 1.035 | .94 | .95 |

As is apparent, like the previous embodiments, a number of these tables corresponding to the different flow settings will be required.

While the invention has been described with reference to particular embodiments, it will be understood by those skilled in the art that various changes may be made and equivalents may be substituted for elements thereof without departing from the scope of the invention. Many modifications may be made to adapt a particular situation or material to the teachings of the invention without departing from the scope of the invention. For example, with a microprocessor having sufficient memory, it is possible to determine the length of the pulse dose by integrating the actual temperatures and pressures. In addition, the invention may incorporate the many of the useful features of the concentrator as disclosed in U.S. Pat. No. 6,764,534.

Therefore, it is intended that the invention not be limited to the particular embodiments disclosed as the best mode contemplated for carrying out this invention, but that the invention will include all embodiments falling within the scope and spirit of the appended claims.

What is claimed is:

1. Apparatus comprising means for producing a product gas having a high concentration of oxygen, means for controlling a desired amount of the product gas delivered through an outlet to a user only on initiation of demand, means for delivering the product gas to the outlet, and means for determining the temperature and/or the pressure of the product gas at or near the output, the control means comprising means utilizing the determined temperature and/or pressure for setting the length of time to supply substantially that amount of product gas to the user.

2. Apparatus according to claim 1 in which the producing means generates the product gas from ambient air at predictable variations in pressure over phases of an operating cycle, and the utilizing means determines the pressure from the phase of the operating cycle at which demand is initiated.

3. Apparatus according to claim 2, in which the control means comprises a set of look-up tables containing the lengths of time specific to the phases of the operating cycle.

4. Apparatus according to claim 3, in which the look-up tables are resident in a microprocessor.

5. Apparatus according to claim 2, in which the producing means generates the product gas by pressure swing adsorption.

6. Apparatus according to claim 1, in which the control means comprises at least one set of look-up tables containing predetermined lengths of time to supply the product gas and specific to a plurality of temperature ranges for each of a plurality of pressure ranges.

7. Apparatus according to claim 6, in which the look-up tables are resident in a microprocessor.

8. Apparatus according to claim 1, in which the control means comprises at least one set of look-up tables containing predetermined lengths of time to supply the product gas and specific to a plurality of pressure ranges.

9. Apparatus according to claim 8, in which the look-up tables are resident in a microprocessor.

10. Apparatus according to claim 1, in which the control means comprises at least one set of look-up tables containing predetermined lengths of time to supply the product gas and specific to a plurality of temperature ranges.

11. Apparatus according to claim 10, in which the look-up tables are resident in a microprocessor.

12. A method for determining a desired dose of a product gas generated by an apparatus having means for producing a high concentration of oxygen and delivering the gas through an outlet to a user, comprising the steps of sensing inhalation by the user, determining the temperature and/or pressure of the product gas at the time inhalation is sensed, and delivering the desired dose of product gas to the user in a length of time based on the determined temperature and/or pressure.

13. The method of claim 12 in which the step of delivering the desired dose of product gas is preceded by the steps of calculating the times required to deliver the desired dose of product gas for the times demand may be initiated, and producing a reference table of the calculated times to be accessed for the delivery step.

14. The method according to claim 13, in which the calculation comprises the steps of dividing the cycle into a plurality of predetermined ranges, supplying the product gas for an arbitrary fixed time to measure at each of the cycle ranges both the actual pressure and the actual volume of product gas delivered during the fixed time, recalculating said gas delivery time at each such cycle range to deliver the desired dose of product gas when demand is initiated at that point in the cycle, and creating the reference table from the cycle ranges and recalculated times.

15. In an apparatus having means for producing doses of product gas having a high concentration of oxygen and means for controlling the supply of the dose to a user only on initiation of demand, the means for generating the product gas having variations in pressure in substantially consistent and sequential operating cycles, a method of determining for the apparatus the length of time required to supply desired and substantially uniform doses to the user, comprising the steps of dividing the overall time of the operating cycle into predetermined ranges, supplying the product gas for an arbitrary fixed time to measure at each of the cycle ranges both the actual pressure and the actual volume of product gas delivered during the fixed time, recalculating the gas delivery time for each such cycle range to deliver the desired dose of product gas when demand is initiated at that cycle range, and producing a reference table by which to read the appropriate delivery times from among the cycle ranges.

16. In an apparatus having means for producing a desired dose of product gas having a high concentration of oxygen and delivering the gas at varying pressures and temperatures through an outlet to a user, a method comprising the steps of sensing inhalation by the user, determining the temperature of the gas supplied at the time at which the inhalation is sensed, determining the pressure of the gas at the time at which the inhalation is sensed, and delivering the gas to the user for a length of time based on the determined temperature and pressure.
